(19) United States
(12) Patent Application Publication
(10) Pub. No.: US 2025/0281045 A1
(43) Pub. Date: Sep. 11, 2025

(54) A SYSTEM CONFIGURED FOR REMOTE MONITORING OF HEART FAILURE PATIENTS

(71) Applicant: Medtronic, Inc., Minneapolis, MN (US)

(72) Inventors: Val D. Eisele, III, Little Canada, MN (US); Juliana E. Pronovici, New Hope, MN (US); Ryan D. Wyszynski, Oak Grove, MN (US); Shantanu Sarkar, Roseville, MN (US); Matthew J. Hoffman, St. Paul, MN (US); Amanda D. Taylor, Roseville, MN (US); David Lanctin, Minneapolis, MN (US); Steven G. Nelson, Raynham, MA (US)

(21) Appl. No.: 18/858,632

(22) PCT Filed: Apr. 12, 2023
(86) PCT No.: PCT/IB2023/053754

§ 371 Date: Oct. 21, 2024

Related U.S. Application Data
(60) Provisional application No. 63/363,454, filed on Apr. 22, 2022.

(51) Int. Cl. A61B 5/00; G06T 7/00; G16H 50/20; G16H 50/30

(52) U.S. Cl. CPC A61B 5/0044; A61B 5/4836; G06T 7/0012; G16H 50/20; G16H 50/30; G06T 2207/10004; G06T 2207/10024; G06T 2207/20081; G06T 2207/30048

(57) ABSTRACT

This disclosure is directed to devices, systems, and techniques for monitoring a status of a health failure patient. An example system is configured to capture one or more images of a region of interest of the patient via the camera sensor. The system is configured to process the one or more images. The system is configured to determine a result based on the processing, the result being related to the heart failure status of the patient. The system is configured to generate a first indication for output regarding the result.

This application claims the benefit of U.S. Provisional Patent Application Ser. No. 63/363,454, filed Apr. 22, 2022, the entire content of which is incorporated herein by reference.

### TECHNICAL FIELD

The disclosure relates generally to devices and, more particularly, devices configured to monitor patient parameters.

### BACKGROUND

Some types of devices may be used to monitor one or more physiological parameters of a patient. Such devices may include, or may be part of a system that includes, sensors that sense signals associated with such physiological parameters. Values determined based on such signals may be used to assist in detecting changes in patient conditions, in evaluating the efficacy of a therapy, or in generally evaluating patient health.

### SUMMARY

Acute decompensated heart failure is a manifestation of worsening of heart failure (or broadly chronic illness) symptoms that may require heart failure hospital admission to relieve such patients of congestion and/or shortness of breath symptoms. Edema, tissue oxygenation, or perfusion may be important physiological parameters to a heart disease patient. The proactive management of heart failure patients may require multiple in-office visits to visually check peripheral edema, tissue oxygenation, or perfusion with a finger plethysmography which may increase the health care costs to patients and physicians. Such visits may be required for all patients, and therefore may reduce the time clinicians can spend on higher-risk patients and may reduce clinic efficiency.

In general, the disclosure is directed to devices, systems, and techniques for using a system to monitor physiological parameters of a patient, such as a heart failure patient, which may reduce or eliminate the need for in-office visits to visually check on physiological parameters of patients. Sensors may be located within or on external devices (e.g., cellular phones, tablet computers, digital cameras, etc.), implantable medical devices (IMDs), and/or wearable devices, for sensing physiological parameters of a patient.

A camera sensor of an external device may be used by a patient or a caregiver to capture images of a region of interest on the patient. As used herein an “image” may include photograph or a video. These images need not be captured in a clinical setting. For example, they may be captured at the patient's residence. Processing circuitry may process such images to determine an edema result, a tissue oxygenation result, and/or a perfusion result. These results may be output to a clinician to proactively manage heart failure patients remotely. This may reduce time spent by the clinician in performing edema and/or tissue oxygenation or perfusion tests, reduce the time invested in travel by the patient, and improve clinical outcomes for the patient, as such tests may be performed more frequently than in a clinical setting, such as daily.

According to the techniques of this disclosure, an external device, such as a cellular phone, a tablet computer, or a digital camera may capture one or more images. Processing circuitry may process the one or more images. Such processing circuitry may be in the external device, in a cloud computing environment, or a combination thereof. For example, the processing circuitry may compare the one or more images to one or more baseline images, which may be collected in a clinical setting, for example, by a clinician when the patient is relatively healthy. The processing circuitry may determine a result based on the comparison and generate an indication, such as a report, based on the result. In some examples, sensor data from an IMD may trigger these techniques.

For example, the techniques of this disclosure may leverage non-implanted consumer available sensors (e.g., a camera sensor, or a flash together with a camera sensor) to measure plethysmography or peripheral edema metrics unavailable in active implantable medical devices to help inform physician visits before, during, or after a heart failure decomposition event. In some examples, such sensors may be used in combination with information from an IMD. Such techniques may ultimately (through the use of clinical data) reduce the need for clinician visits. For example, one or more camera photos of a patient ankle, neck area (e.g., to determine Jugular Vein Distention), or a hand may be processed through the use of photo recognition, such as a computer vision model, and an algorithm to improve the specificity of existing heart failure risk scores, determine patient improvement following an intervention, or aid in detection of symptom risk in the absence of heart failure (e.g., renal function). The system with the combination of the flash and camera may be used to identify tissue perfusion. The techniques may be triggered by a cloud computing environment, such as CareLink, by an edge computing device, such as a patient's smartphone or other smart device, or directly by the IMD to instruct users to perform this task such that additional data may be collected which may be used to improve specificity of a diagnosis of a client condition (e.g., heart failure). This solution may also be performed on demand for patients so as to track with other data provided to them via their IMD to assist with at home self-management.

In some examples, the techniques and systems of this disclosure may use a machine learning model to more accurately determine a heart failure risk level or other patient condition or status based on image data. In some examples, the machine learning model is trained with a set of training instances, where one or more of the training instances comprise data that indicate relationships between various sets of input data and outputs. Because the machine learning model is trained with potentially thousands or millions of training instances, the machine learning model may reduce the amount of error in risk level or other values useful for control of dialysis. Reducing errors using the techniques of this disclosure may provide one or more technical and clinical advantages, such as improving patient health outcomes.

In some examples, a system for monitoring a heart failure status of a patient includes: memory configured to store one or more images; a camera sensor; and processing circuitry communicatively coupled to the memory, and the camera sensor, the processing circuitry being configured to: capture one or more images of a region of interest of the patient via the camera sensor; process the one or more images; determine a result based on the processing, the result being related to the heart failure status of the patient; and generate a first indication for output regarding the result.

In some examples, a method of monitoring a heart failure status of a patient includes: capturing, by processing circuitry, one or more images of a region of interest of the patient via a camera sensor; processing, by the processing circuitry, the one or more images; determining, by the processing circuitry, a result based on the processing, the result being related to the heart failure status of the patient; and generating, by the processing circuitry, a first indication for output regarding the result.

In some examples, a non-transitory computer-readable medium includes instructions, which when executed, cause processing circuitry to: capture one or more images of a region of interest of interest patient via the camera sensor; process the one or more images; determine a result based on the processing, the result being related to the heart failure status of the patient; and generate a first indication for output regarding the result.

This summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the implantable medical device (IMD) of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B are block diagrams illustrating two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a block diagram illustrating an example system that includes an access point, a network, external computing devices, such as a server, and one or more other computing devices, which may be coupled to the IMD of FIGS. 1-4, an external device, and processing circuitry via a network, in accordance with one or more techniques described herein.

FIG. 7 is a conceptual diagram illustrating the use of photography to determine an edema result.

FIG. 8 is a conceptual diagram illustrating the use of photography to determine an oxygen saturation or perfusion result.

FIG. 9 is a flow diagram illustrating an example of image analysis techniques for monitoring a heart failure status of a patient according to one or more aspects of this disclosure.

FIG. 10 is a conceptual diagram illustrating an example machine learning model configured to determine heart failure risk or status of another patient condition based on image data.

FIG. 11 is a conceptual diagram illustrating an example training process for an artificial intelligence model, in accordance with examples of the current disclosure.

Like reference characters denote like elements throughout the description and figures.

### DETAILED DESCRIPTION

This disclosure describes techniques for remotely monitoring a heart failure status of a patient. These techniques may include having a patient or caregiver take images of a region of interest on the patient, which may be outside of a clinical environment. These images may be processed, e.g., compared to one or more baseline images. The comparison may yield a result which may be indicative of edema of the patient and/or tissue oxygenation or perfusion of the patient.

The techniques of this disclosure may facilitate the remote monitoring and management of a heart failure patient, peripheral vascular disease patient, diabetes patient, patient at risk of deep vein thrombosis, and/or other cardiac patient. By providing for the remote monitoring of edema of the patient and/or tissue oxygenation or perfusion of the patient, the techniques of this disclosure may lead to better clinical outcomes for the patient, improve clinic efficiency, reduce patient travel time, and reduce medical costs.

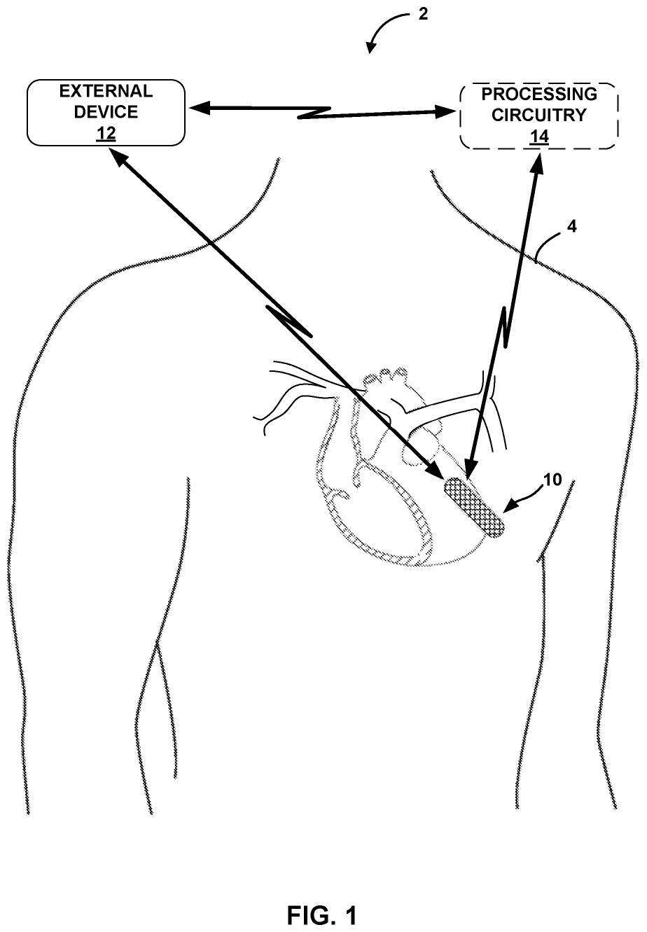

FIG. 1 illustrates the environment of an example medical device system 2 in conjunction with a patient 4, in accordance with one or more techniques of this disclosure. While the techniques described herein are generally described in the context of an insertable cardiac monitor and/or an external device, the techniques of this disclosure may be implemented in any implantable medical device or external device or combination thereof, such as a pacemaker, a defibrillator, a cardiac resynchronization therapy device, an implantable pulse generator, an intra-cardiac pressure measuring device, a ventricular assist device, a pulmonary artery pressure device, a subcutaneous blood pressure device, or an external device having a telemetry system and weight sensors, blood pressure sensors, pulse oximeters, activity sensors, patch systems, or the like, configured to sense physiological parameters of a patient. The example techniques may be used with an IMD 10, which may be in wireless communication with at least one of external device 12 and other devices not pictured in FIG. 1. Processing circuitry 14 is conceptually illustrated in FIG. 1 as separate from IMD 10 and external device 12, but may be processing circuitry of IMD 10 and/or processing circuitry of external device 12. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with IMD 10 and/or external device 12 via a network.

In some examples, IMD 10 is implanted outside of a thoracic cavity of patient 4 (e.g., subcutaneously in the pectoral location illustrated in FIG. 1). IMD 10 may be positioned near the sternum near or just below the level of patient 4's heart, e.g., at least partially within the cardiac silhouette. For other medical conditions, IMD 10 may be implanted in other appropriate locations, such as the interstitial space, abdomen, back of arm, wrist, etc. In some examples, IMD 10 takes the form of a Reveal LINQ™ or LINQ II™ Insertable Cardiac Monitor (ICM), available from Medtronic, Inc., of Minneapolis, Minnesota.

Clinicians sometimes diagnose patients with medical conditions based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patient is in a clinic for a medical appointment. However, in some examples, physiological markers (e.g., arrhythmia, etc.) of a patient condition are rare or are difficult to observe over a relatively short period of time. As such, in these examples, a clinician may be unable to observe the physiological markers needed to diagnose a patient with a medical condition or effectively treat the patient while monitoring one or more physiological signals of the patient during a medical appointment. Moreover, even if the clinician is able to observe some physiological markers, such as edema and/or oxygen saturation or perfusion during a medical appointment, such medical appointments may be relatively separated in time, such that worsening edema and/or oxygen saturation or perfusion may not be easily monitored.

In the example illustrated in FIG. 1, IMD 10 is implanted within patient 4 to continuously record one or more physiological signals, such as an electrocardiogram (ECG), electromyogram (EMG), impedance, respiration, activity, posture, blood oxygen saturation, or other physiological signals, of patient 4 over an extended period of time. In some examples, IMD 10 includes a plurality of electrodes. The plurality of electrodes is configured to detect signals that enable processing circuitry 14, e.g., of IMD 10, to monitor and/or record physiological parameters of patient 4. For example, the plurality of electrodes may be configured to sense an ECG, EMG, impedance, or the like, of patient 4. IMD 10 may additionally or alternatively include one or more optical sensors, accelerometers, temperature sensors, chemical sensors, light sensors, pressure sensors, and/or respiratory sensors, in some examples. Such sensors may detect one or more physiological parameters indicative of a patient condition. The physiological parameters may include impedance, such as interstitial or other impedance indicative of fluid accumulation analyzed according to the OptiVol™ algorithm or another algorithm that determines a fluid index based on impedance, patient activity, anti-tachycardia/anti-fibrillation burden, ventricular rate during anti-tachycardia/anti-fibrillation, percentage of ventricular pacing, shocks, treated ventricular tachycardia/ventricular fibrillation, night ventricular rate, heart rate variability, heart sounds, lung sounds, and/or other physiological parameters. In some examples, additional sensors may be located on other devices (not shown in FIG. 1) which may also sense physiological parameters of patient 4.

Sensor data may be collected by various devices such as implantable therapy devices, implantable monitoring devices, wearable devices, point of care devices, and noncontact sensors in the home or vehicle or other area frequented by the patient or a combination of such sensor platforms. The sensor data collected may be relevant to the disease state (e.g., heart failure) or comorbidities (e.g., chronic obstructive pulmonary disease (COPD), kidney disease, etc.) or for diagnosing a suspected comorbidity. For patients with multiple comorbidities, it may be possible to perform a differential diagnosis between different sources of a problem, e.g., heart failure decompensation, COPD exacerbation, pneumonia, etc. This would permit a clinician to prescribe appropriate therapies to address the patient's condition, such as diuretics, antibiotics, encourage fluids, etc.

Processing circuitry 14 may monitor such sensed physiological parameters as impedance (e.g., using a fluid index algorithm), patient activity, atrial tachycardia (AT)/atrial fibrillation (AF) burden, ventricular rate during AT/AF, percentage of ventricular pacing, shocks, treated ventricular tachycardia/ventricular fibrillation, night ventricular rate, heart rate variability, heart sounds, lung sounds, and/or other physiological parameters. For example, processing circuitry 14 may derive features (such as a fluid index) from aggregated data. Raw data, aggregated data, and determined physiological features may be applied to artificial intelligence algorithms or machine learning models, e.g., deep learning models, to generate clinically actionable insights. In some examples, the sensed physiological parameters may be collected and processed to provide an index or score of patient 4's health. There are several examples of such scoring systems that are in clinical use today. One example is the APACHE (Acute Physiology and Chronic Health Evaluation) IV scoring system. In some examples, processing circuitry 14 may continuously calculate such a score, or a similar score. Processing circuitry 14 may attempt to determine any source of changes in patient 4's health based on such sensed physiological parameters and, when appropriate, send an indication to a clinician regarding the change in the score and the source of the changes in the patient's health status with suggested treatment options. Processing circuitry 14 may also provide to patient 4 or a caregiver of patient 4 any prescription changes in medications or instructions to consume more liquid. These scores and directions could be provided via external device 12.

However, IMD 10 may not be configured to determine an edema status, an oxygen saturation status, or a perfusion status of patient 4, or in the case where IMD 10 may be configured to determine an edema status, an oxygen saturation status or a perfusion status of patient 4, system 2 may benefit from an additional manner of determining such statuses and using both to determine a heart failure status of patient 4. For example, such a second determination may be used to confirm a heart failure status of patient 4. In such a case, external device 12 may capture one or more images of a region of interest on patient 4. For example, patient 4 or a caregiver may operate external device to capture the one or more images. The region of interest may be on an ankle of patient 4, a hand of patient 4, a leg of patient 4, a foot of patient 4, a face of patient 4, a neck of patient 4, an eye of patient 4, an eyelid of patient 4, or the like. For example, an ankle, hand, leg, foot, face, or neck of patient 4 may be used to determine edema, tissue oxygenation, or perfusion. An eye may be used to determine eye color or glassiness (e.g., reflectivity), or to determine changes from one or more reference images, such as changes in the color of a sclera or iris, changes in clarity of an eye lens, changes in glassiness, changes in presence or prominence of vasculature, changes in evidence of emboli, changes in the color of a tear duct which may be indicative of cardiovascular health. An eye may also be used to determine changes between sequential captured images in a pupil response to a flash, eye tracking a moving image on the screen, etc. which may be indicative of cardiovascular health. A lower eyelid may be used to determine lower eyelid color which may be indicative of cardiovascular health (e.g., a red lower eyelid may signify good cardiovascular health). In some examples, rather than the eye being the region of interest, the region of interest may be the face and processing circuitry 14 may extract from one or more captured images one or both eyes of patient 4 for such determinations.

Processing circuitry 14, which may be within external device 12, a cloud computing environment, or both, may process the one or more images. For example, processing circuitry 14 may compare the one or more images to one or more baseline images. Processing circuitry 14 may determine a result based on the comparison. This result may be related to the heart failure status of patient 4. Processing circuitry 14 may generate an indication for output regarding the result. For example, processing circuitry may output the indication via external device 12 and/or another external device not depicted in FIG. 1.

Processing circuitry 14 may receive sensor data. For example, processing circuitry 14 may receive sensor data from IMD 10 or other sensors in or about patient 4. In some examples, some or all of the sensor data may act as a trigger that triggers external device 12 to inform patient 4 or a caregiver that patient 4 or the caregiver should use external device 12 to capture the one or more images.

External device 12 may be a hand-held computing device with a display viewable by the user and an interface for providing input to external device 12 (i.e., a user input mechanism). For example, external device 12 may include a small display screen (e.g., a liquid crystal display (LCD) or a light emitting diode (LED) display) that presents information to the user, such as instructions to capture the one or more images. External device 12 may include a camera sensor configured to capture the one or more images. In some examples, external device 12 may also include a flash for use with the camera sensor.

In addition, external device 12 may include a touch screen display, keypad, buttons, a peripheral pointing device, voice activation, or another input mechanism that allows the user to navigate through the user interface of external device 12 and provide input. For example, external device 12 may pose questions to a user, such as patient 4 related to symptoms patient 4 may be experiencing and the user may provide answers to such questions via the user interface so that external device 12 may gather further information relating to the heart failure status of patient 4. If external device 12 includes buttons and a keypad, the buttons may be dedicated to performing a certain function, e.g., a power button, the buttons and the keypad may be soft keys that change in function depending upon the section of the user interface currently viewed by the user, or any combination thereof.

In some examples, external device 12 may be a separate application within another multi-function device, rather than a dedicated computing device. For example, the multi-function device may be a cellular phone, a tablet computer, a digital camera, or another computing device that may run an application that enables external device to operate as described herein.

When external device 12 is configured for use by the clinician, external device 12 may be used to transmit instructions to IMD 10 and to receive measurements, such sensed physiological parameters, heart failure scores, or the like. Example instructions may include requests to set electrode combinations for sensing and any other information that may be useful for programming into IMD 10. The clinician may also configure and store operational parameters for IMD 10 within IMD 10 with the aid of external device 12. In some examples, external device 12 assists the clinician in the configuration of IMD 10 by providing a system for identifying potentially beneficial operational parameter values.

Whether external device 12 is configured for clinician or patient use, external device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

Processing circuitry 14, in some examples, may include one or more processors that are configured to implement functionality and/or process instructions for execution within IMD 10 and/or external device 12. For example, processing circuitry 14 may be capable of processing instructions stored in a storage device. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

Processing circuitry 14 may represent processing circuitry located within any combination of IMD 10 and/or external device 12. In some examples, processing circuitry 14 may be entirely located within a housing of IMD 10. In other examples, processing circuitry 14 may be entirely located within a housing of external device 12. In other examples, processing circuitry 14 may be located within any combination of IMD 10, external device 12, and another device or group of devices that are not illustrated in FIG. 1. As such, techniques and capabilities attributed herein to processing circuitry 14 may be attributed to any combination of IMD 10, external device 12, and other devices that are not illustrated in FIG. 1.

In some examples, IMD 10 includes one or more motion sensors, such as accelerometers. An accelerometer of IMD 10 may collect an accelerometer signal which reflects a measurement of a motion of patient 4. In some cases, the accelerometer may collect a three-axis accelerometer signal indicative of patient 4's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 4 along a vertical axis, the lateral axis accelerometer signal vector may represent an acceleration of patient 4 along a lateral axis, and the frontal axis accelerometer signal vector may represent an acceleration of patient 4 along a frontal axis. In some cases, the vertical axis substantially extends along a torso of patient 4 when patient 4 from a neck of patient 4 to a waist of patient 4, the lateral axis extends across a chest of patient 4 perpendicular to the vertical axis, and the frontal axis extends outward from and through the chest of patient 4, the frontal axis being perpendicular to the vertical axis and the lateral axis. In some examples, processing circuitry 14 may be configured to identify, based on one of more accelerometer signals, a posture or activity of patient 4. In some examples, the sensed physiological parameters by IMD 10 may include the posture and/or activity of patient 4. Such postures and/or activities of patient 4 may be used, together with other sensor signals, when determining a heart failure score.

Although in one example IMD 10 takes the form of an ICM, in other examples, IMD 10 takes the form of any one or more of an ICM, a pacemaker, a defibrillator, a cardiac resynchronization therapy device, an implantable pulse generator, an intra-cardiac pressure measuring device, a ventricular assist device, a pulmonary artery pressure device, a subcutaneous blood pressure device, or the like. The physiological parameters may be sensed or determined using one or more of the aforementioned devices, as well as external devices such as external device 12.

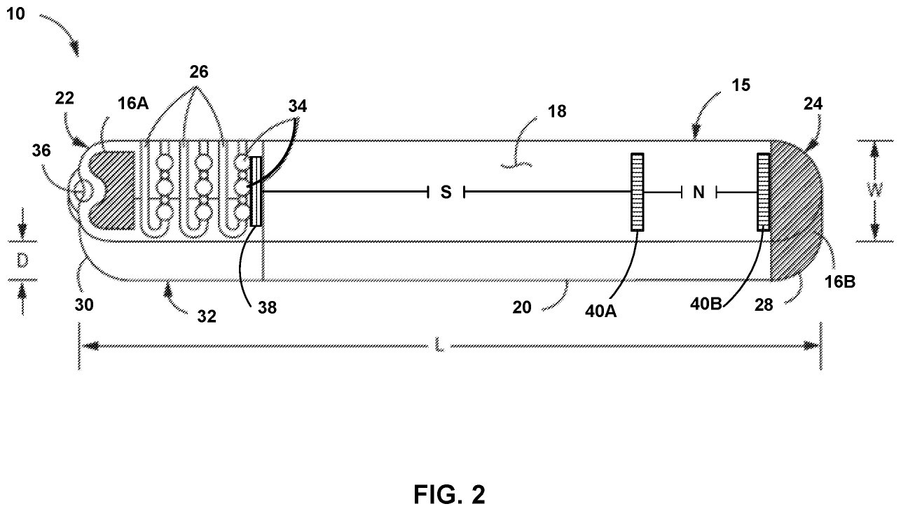

FIG. 2 is a conceptual drawing illustrating an example configuration of IMD 10 of the medical device system 2 of FIG. 1, in accordance with one or more techniques described herein. In the example shown in FIG. 2, IMD 10 may be a leadless, vascularly-implantable monitoring device having housing 15, proximal electrode 16A, and distal electrode 16B. Housing 15 may further include first major surface 18, second major surface 20, proximal end 22, and distal end 24. In some examples, IMD 10 may include one or more additional electrodes 16C, 16D positioned on one or both of major surfaces 18, 20 of IMD 10. Housing 15 encloses electronic circuitry located inside the IMD 10, and protects the circuitry contained therein from fluids such as body fluids (e.g., blood). In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted under the skin of the patient using a minimally invasive procedure and to remain in the desired orientation during insertion. In addition, IMD 10 may include radial asymmetries (e.g., the rectangular shape) along a longitudinal axis of IMD 10, which may help maintain the device in a desired orientation following implantation.

In some examples, a spacing between proximal electrode 16A and distal electrode 16B may range from about 30-55 mm, about 35-55 mm, or about 40-55 mm, or more generally from about 25-60 mm. Overall, IMD 10 may have a length L of about 20-30 mm, about 40-60 mm, or about 45-60 mm. In some examples, the width W of major surface 18 may range from about 3-10 mm, and may be any single width or range of widths between about 3-10 mm. In some examples, a depth D of IMD 10 may range from about 2-9 mm. In other examples, the depth D of IMD 10 may range from about 2-5 mm, and may be any single or range of depths from about 2-9 mm. In any such examples, IMD 10 is sufficiently compact to be implanted within the subcutaneous space of patient 4 in the region of a pectoral muscle.

IMD 10, according to an example of the present disclosure, may have a geometry and size designed for ease of implant and patient comfort. Examples of IMD 10 described in this disclosure may have a volume of 3 cubic centimeters (cm³) or less, 1.5 cm³ or less, or any volume therebetween. In addition, in the example shown in FIG. 2, proximal end 22 and distal end 24 are rounded to reduce discomfort and irritation to surrounding tissue once implanted under the skin of patient 4.

In the example shown in FIG. 2, first major surface 18 of IMD 10 faces outward towards the skin, when IMD 10 is inserted within patient 4, whereas second major surface 20 is faces inward toward musculature of patient 4. Thus, first and second major surfaces 18, 20 may face in directions along a sagittal axis of patient 4 (see FIG. 1), and this orientation may be generally maintained upon implantation due to the dimensions of IMD 10.

Proximal electrode 16A and distal electrode 16B may be used to sense cardiac EGM signals (e.g., ECG signals) when IMD 10 is implanted subcutaneously in patient 4. In some examples, processing circuitry of IMD 10 also may determine whether cardiac ECG signals of patient 4 are indicative of arrhythmia or other abnormalities, which processing circuitry of IMD 10 may evaluate in determining whether a medical condition (e.g., heart failure, sleep apnea, or COPD) of patient 4 has changed. The cardiac ECG signals may be stored in a memory of IMD 10, and data derived from the cardiac ECG signals and/or other sensor signals, such as an impending heart failure decompensation event may be transmitted via integrated antenna 26 to another device, such as external device 12. Additionally, in some examples, electrodes 16A, 16B may be used by communication circuitry of IMD 10 for tissue conductance communication (TCC) communication with external device 12 or another device.

In the example shown in FIG. 2, proximal electrode 16A is in close proximity to proximal end 22, and distal electrode 16B is in close proximity to distal end 24 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer (e.g., external device 12) via inductive coupling, electromagnetic coupling, tissue conductance, Near Field Communication (NFC), Radio Frequency Identification (RFID), Bluetooth®, WiFi®, or other proprietary or non-proprietary wireless telemetry communication schemes. Antenna 26 may be coupled to communication circuitry of IMD 10, which may drive antenna 26 to transmit signals to external device 12, and may transmit signals received from external device 12 to processing circuitry of IMD 10 via communication circuitry.

In some examples, IMD 10 may include several features for retaining IMD 10 in position once subcutaneously implanted in patient 4, so as to decrease the chance that IMD 10 migrates in the body of patient 4. For example, as shown in FIG. 2, housing 15 may include anti-migration projections 34 positioned adjacent integrated antenna 26. Anti-migration projections 34 may include a plurality of bumps or protrusions extending away from first major surface 18, and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 includes suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10.

In the example shown in FIG. 2, IMD 10 includes a light emitter 38, a proximal light detector 40A, and a distal light detector 40B positioned on housing 15 of IMD 10. Light detector 40A may be positioned at a distance S from light emitter 38, and a distal light detector 40B positioned at a distance S+N from light emitter 38. In other examples, IMD 10 may include only one of light detectors 40A, 40B, or may include additional light emitters and/or additional light detectors. Although light emitter 38 and light detectors 40A, 40B are described herein as being positioned on housing 15 of IMD 10, in other examples, one or more of light emitter 38 and light detectors 40A, 40B may be positioned, on a housing of another type of IMD within patient 4, such as a transvenous, subcutaneous, or extravascular pacemaker or ICD, or connected to such a device via a lead.

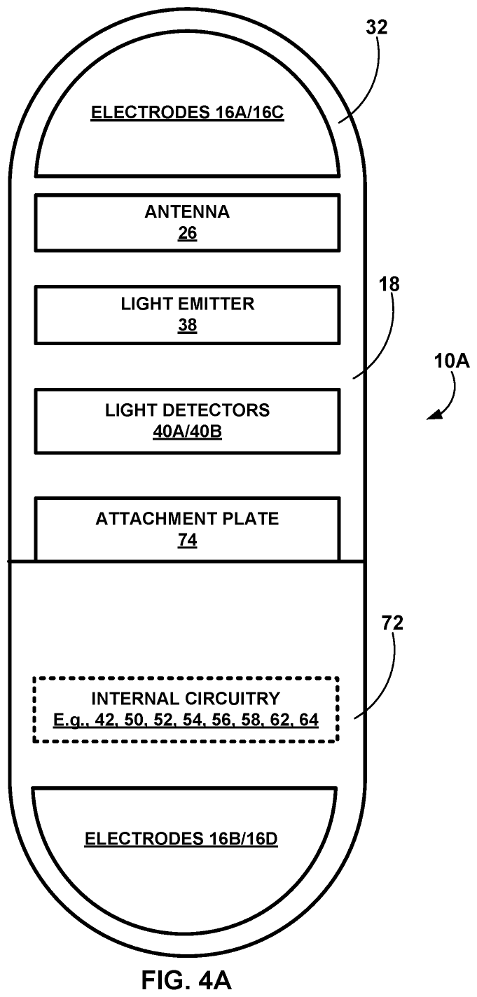

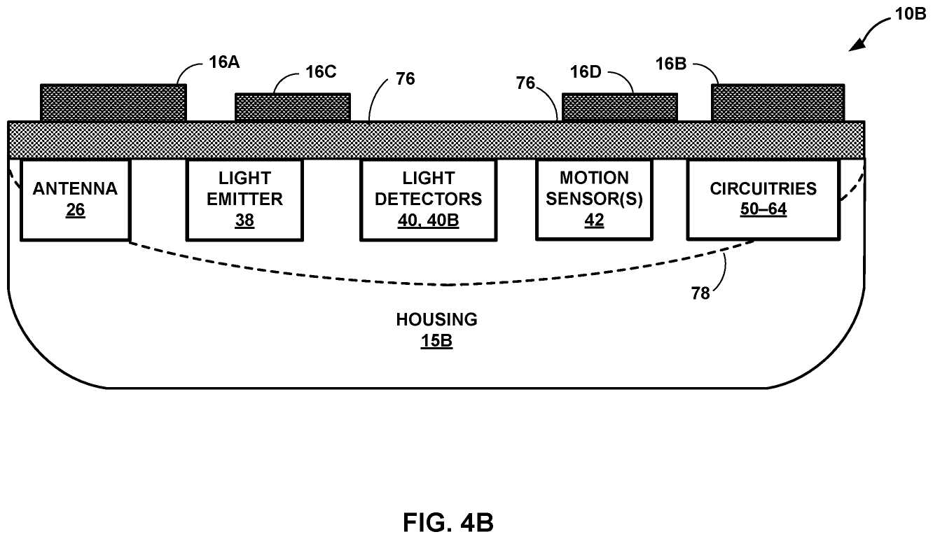

As shown in FIG. 2, light emitter 38 may be positioned on header assembly 32, although, in other examples, one or both of light detectors 40A, 40B may additionally or alternatively be positioned on header assembly 32. In some examples, light emitter 38 may be positioned on a medial section of IMD 10, such as part way between proximal end 22 and distal end 24. Although light emitter 38, light detectors 40A, 40B, and sensors 44 are illustrated as being positioned on first major surface 18, light emitter 38, light detectors 40A, 40B, and sensors 44 alternatively may be positioned on second major surface 20. In some examples, IMD may be implanted such that light emitter 38 and light detectors 40A, 40B face inward when IMD 10 is implanted, toward the muscle of patient 4, which may help minimize interference from background light coming from outside the body of patient 4. Light detectors 40A, 40B may include a glass or sapphire window, such as described below with respect to FIG. 4B, or may be positioned beneath a portion of housing 15 of IMD 10 that is made of glass or sapphire, or otherwise transparent or translucent.

In some examples, IMD 10 may include one or more additional sensors, such as one or more motion sensors (not shown in FIG. 2). Such motion sensors may be 3D accelerometers configured to generate signals indicative of one or more types of movement of the patient, such as gross body movement (e.g., motion) of the patient, patient posture, movements associated with the beating of the heart, or coughing, rales, or other respiration abnormalities, or the movement of IMD 10 within the body of patient 4. One or more of the parameters monitored by IMD 10 (e.g., bio impedance, respiration rate, EGM, etc.) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. Thus, in some methods of identifying or tracking a medical condition of patient 4, it may be advantageous to account for such fluctuations when determining whether a change in a parameter is indicative of a change in a medical condition or when determining a heart failure score.

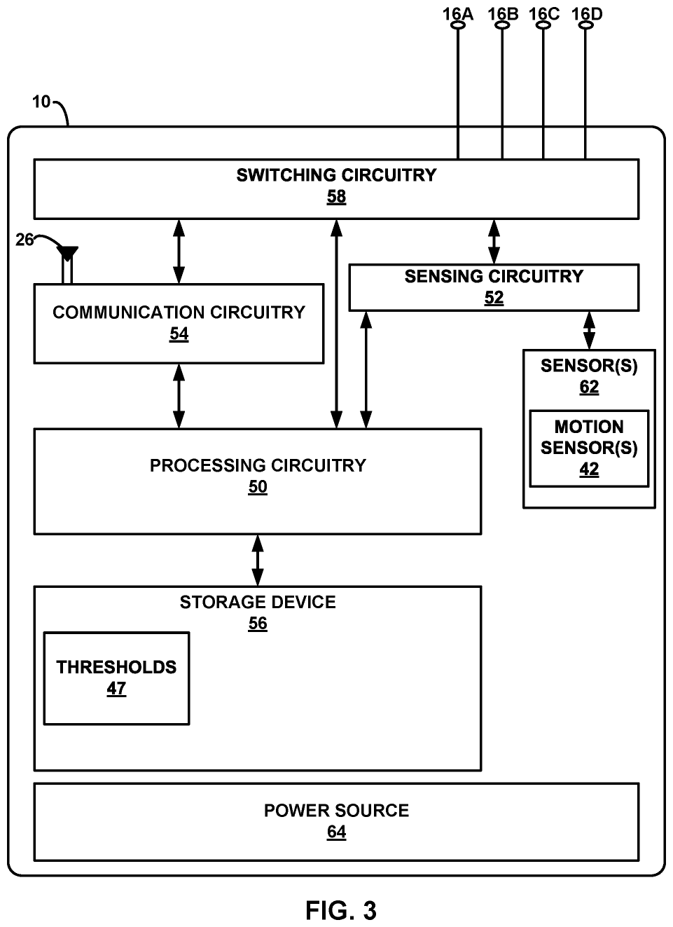

FIG. 3 is a functional block diagram illustrating an example configuration of IMD 10 of FIGS. 1 and 2, in accordance with one or more techniques described herein. In the illustrated example, IMD 10 includes electrodes 16, antenna 26, processing circuitry 50, sensing circuitry 52, communication circuitry 54, storage device 56, switching circuitry 58, sensors 62 including motion sensor(s) 42 (which may include an accelerometer), and power source 64.

Processing circuitry 50 may include fixed function circuitry and/or programmable processing circuitry. Processing circuitry 50 may include any one or more of a microprocessor, a controller, a DSP, an ASIC, an FPGA, or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof. In some examples, one or more techniques of this disclosure may be performed by processing circuitry 50.

Sensing circuitry 52 and communication circuitry 54 may be selectively coupled to electrodes 16A-16D via switching circuitry 58, as controlled by processing circuitry 50. Sensing circuitry 52 may monitor signals from electrodes 16A-16D in order to monitor electrical activity of heart (e.g., to produce an ECG). Sensing circuitry 52 also may monitor signals from sensors 62, which may include motion sensor(s) 42 (which may include an accelerometer) and sensors 44. In some examples, sensing circuitry 52 may include one or more filters and amplifiers for filtering and amplifying signals received from one or more of electrodes 16A-16D and/or motion sensor(s) 42.

Communication circuitry 54 may include any suitable hardware, firmware, software or any combination thereof for communicating with another device, such as external device 24 or another IMD or sensor, such as a pressure sensing device. Under the control of processing circuitry 50, communication circuitry 54 may receive downlink telemetry from, as well as send uplink telemetry to, external device 12 or another device with the aid of an internal or external antenna, e.g., antenna 26. In addition, processing circuitry 50 may communicate with a networked computing device via an external device (e.g., external device 24) and a computer network, such as the Medtronic CareLink® Network developed by Medtronic, plc, of Dublin, Ireland.

A clinician or other user may retrieve data from IMD 10 using external device 12, or by using another local or networked computing device configured to communicate with processing circuitry 50 via communication circuitry 54. The clinician may also program parameters of IMD 10 using external device 12 or another local or networked computing device.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), ferroelectric RAM (FRAM) read-only memory (ROM), non-volatile RAM (NVRAM), electrically-erasable programmable ROM (EEPROM), flash memory, or any other digital media.

Power source 64 is configured to deliver operating power to the components of IMD 10. Power source 64 may include a battery and a power generation circuit to produce the operating power. In some examples, the battery is rechargeable to allow extended operation. In some examples, recharging is accomplished through proximal inductive interaction between an external charger and an inductive charging coil within external device 12. Power source 64 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

FIGS. 4A and 4B illustrate two additional example IMDs that may be substantially similar to IMD 10 of FIGS. 1-3, but which may include one or more additional features, in accordance with one or more techniques described herein. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72, an attachment plate 74, and treatment 45. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, storage device 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D, light detectors 40A, 40B on housing 15B and processing circuitry 50. In some examples, insulative cover 76 may be positioned over an open housing 15 to form the housing for the components of IMD 10B. One or more components of IMD 10B (e.g., antenna 26, light emitter 38, light detectors 40A, 40B, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B. When flipped and placed onto housing 15B, the components of IMD 10B formed on the bottom side of insulative cover 76 may be positioned in a gap 78 defined by housing 15B.

Insulative cover 76 may be configured so as not to interfere with the operation of IMD 10B. For example, one or more of electrodes 16A-16D may be formed or placed above or on top of insulative cover 76, and electrically connected to switching circuitry 58 through one or more vias (not shown) formed through insulative cover 76. Insulative cover 76 may be formed of sapphire (i.e., corundum), glass, parylene, and/or any other suitable insulating material. Sapphire may be greater than 80% transmissive for wavelengths in the range of about 300 nm to about 4000 nm, and may have a relatively flat profile. In the case of variation, different transmissions at different wavelengths may be compensated for, such as by using a ratiometric approach. In some examples, insulative cover 76 may have a thickness of about 300 micrometers to about 600 micrometers. Housing 15B may be formed from titanium or any other suitable material (e.g., a biocompatible material), and may have a thickness of about 200 micrometers to about 500 micrometers. These materials and dimensions are examples only, and other materials and other thicknesses are possible for devices of this disclosure.

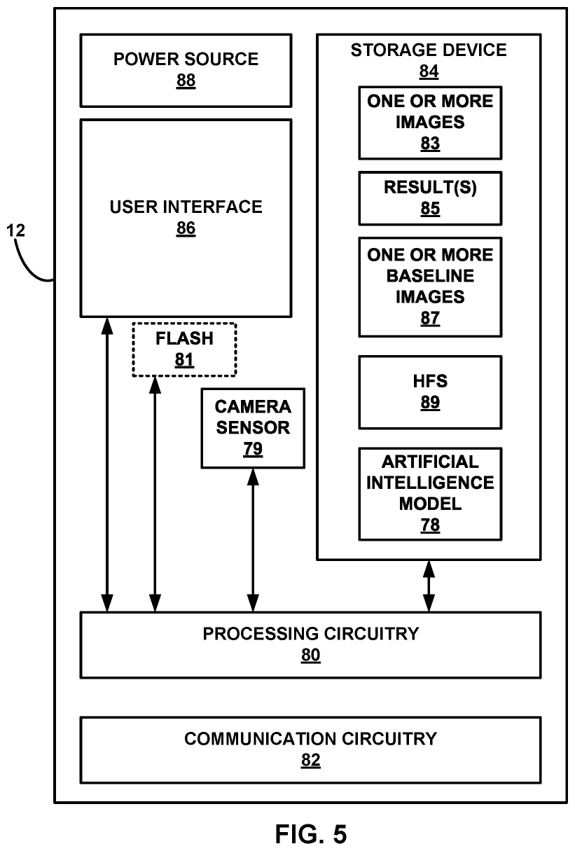

FIG. 5 is a block diagram illustrating an example configuration of components of external device 12, in accordance with one or more techniques of this disclosure. In the example of FIG. 5, external device 12 includes processing circuitry 80, camera sensor 79, communication circuitry 82, storage device 84, user interface 86, power source 88, and, in some examples, flash 81.

Processing circuitry 80, in one example, may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80. In some examples, processing circuitry 80 may perform one or more of the techniques of this disclosure.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, dynamic random access memories (DRAM), static random access memories (SRAM), magnetic discs, optical discs, flash memories, or forms of electrically programmable memories (EPROM) or EEPROM. In some examples, storage device 84 is used to store data indicative of instructions for execution by processing circuitry 80. Storage device 84 may be used by software or applications running on external device 12 to temporarily store information during program execution.

Storage device 84 may also store artificial intelligence model 78, one or more images 83, result(s) 85, and one or more baseline images 87. In some examples, artificial intelligence model 78 may be used by processing circuitry 80 to process one or more images 83. For example, processing circuitry may execute artificial intelligence model 78 to compare one or more images 83 to one or more baseline images 87. In some examples, artificial intelligence model 78 may include a computer vision model that may be trained on images from a plurality of patients relating to edema, tissue oxygenation, and/or perfusion. In some examples, the computer vision model may further be trained on clinicians' notes, scores, determined results, rankings, or the like applied to the images from the plurality of patients. In this manner, artificial intelligence model 78 may, when executed by processing circuitry 80, cause processing circuitry 80 to determine a result without further input from a clinician. Processing circuitry 80 may store the result in result(s) 85 in storage device 84.

For example, processing circuitry 80 executing artificial intelligence model 78 may analyze one or more images 83 and one or more baseline images 87 to determine differences therebetween and apply labels to such differences. In some examples, artificial intelligence model may include a machine learning model, such as a k-means clustering model.

For example, a k-means clustering model may be used having a plurality of clusters, each associated with a different ranking of heart failure status. One or more images 83 and one or more baseline images 87 may be compared and the results of the comparison may be associated with a vector that includes variables for, e.g., differences, similarities, number of differences, number of similarities, etc. The location of the vector in a given one of the clusters may be indicative of a heart failure ranking. Processing circuitry 80 may use such a heart failure ranking to update or modify a calculated heart failure score based on sensor signals from IMD 10, for example. Other potential machine learning techniques include Naïve Bayes, k-nearest neighbors, random forest, support vector machines, neural networks, linear regression, logistic regression, etc.

For example, processing circuitry 80 may compare one or more images 83 to one or more baseline images 87. Processing circuitry 80 may determine a result based on the comparison. This result may be an edema result, a tissue oxygenation result, or a perfusion result and may be related to the heart failure status of patient 4. Processing circuitry 80 may generate an indication for output regarding the result. For example, the indication may include the one or more images, a ranking, a score (e.g., a heart failure score), and/or the like.

A patient or a clinician may take one or more images during a clinician visit to determine a “baseline” of a region of interest. In some examples, the one or more images may include a cluster of images. For example, a single press of a button may capture a plurality of images. Such one or more images may be taken when the patient or the clinician applies pressure to the region of interest and then releases the pressure. Images may be taken throughout the process of applying pressure and releasing the pressure. The response of the tissue to pressure may be indicative of swelling of the region of interest of the patient. Processing circuitry of a clinician device or a server, such as a server, for example, in a cloud computing environment, may process these one or more baseline images to create one or more baseline images 87. For example, processing circuitry may execute an artificial intelligence model, such as artificial intelligence model 78 to determine features of the one or more baseline images and determine one or more baseline images 87. These one or more baseline images, in addition to ongoing images overtime may help establish a baseline, which processing circuitry 80 may use to identify relatively large deflections (e.g., deltas) from the baseline line. In some examples, the one or more baseline images may also be taken during a hospitalization or heart failure decompensation event prior to drug or surgical intervention to assist with patient recovery.

In some examples, patient 4 may be requested by external device 12 on a periodic bases, e.g., at least once a day, or at the request of a caregiver, or IMD 10 to take one or more images 83 of a region of interest on their anatomy, such as on their ankle. One or more images 83 may be collected under which the patient would apply pressure to the area with a hand not being used to hold external device 12. Processing circuitry 80 may then process one or more images 83 through the use of an algorithm, such as artificial intelligence model 78, to identify critical time points are identified (1) pre-touch, (2) during touch, (3) post touch.

In some examples, mobility of patient 4 may be reduced, which may lead to less-than-optimal images. In such examples, camera sensor 79 may capture one or more images of region of interest of another, easier to access, extremity. In some examples, camera sensor 79 is a high-resolution camera, which may aid in patient 4 taking better quality images via camera sensor 79. In the example where external device 12 represents a cellular phone, camera sensor 79 may have a 1000×2000 resolution, which may make it possible for processing circuitry 80 to post process one or more images 83 to improve the image quality and thereby possibly improve the accuracy of the techniques of this disclosure.

Processing circuitry 80 may compare one or more images 83 to previously collected one or more baseline images by comparing multiple points in the images to determine a change in size (indicative of swelling) and tissue response. For example, a slow color change of the tissue may be indicative of above normal edema. In some examples, the processing may occur in external device 12, in a cloud computing environment, or a combination thereof. The results may be sent to the patient to communicate, for example, “good” or “poor” results to encourage the patient to take their fluid medication. Processing circuitry 80 may record the results and generate an indication for output to the clinician, patient 4, and/or a caregiver. In some examples, the results may include alphabetic rankings, numerical rankings, color coded rankings (e.g., green, yellow, or red), or the like. Such rankings may be indicative of whether the result is good or poor.

In another example, the results may be integrated into a larger risk-based solution including data from IMD 10, e.g., values of one or more physiological parameters, to help reduce unnecessary treatment. In some examples, the results and data from IMD 10 may be used for a “differential diagnosis.” Not all peripheral swelling is tied to heart failure decompensation, if there is significant swelling in the absence of IMD 10 supporting a hospitalization risk, a clinician may want to intervene.

Alternatively, or additionally, processing circuitry 80 may use the results to provide more specificity in a heart failure score 89 by executing a heart failure algorithm (not shown). Such heart failure algorithm may provide a prediction of a heart failure event and/or a prediction of recovery from a heart failure event. For example, processing circuitry 80 may execute the heart failure algorithm to calculate a heart failure score (e.g., an APACHE IV score) based on sensed physiological signals from IMD 10. Processing circuitry 80 may use the results to modify the calculated heart failure score. For example, if the results show a higher likelihood of a heart failure decomposition event than the calculated heart failure score indicates, processing circuity 80 may increase the heart failure decomposition score by an amount based on the higher likelihood or replace the calculated heart failure score with one supported by the results.

For use of external device 12 for plethysmography, a patient baseline may be established during periods of relatively good health via one or more images collected in the steps below. A baseline may be determined to show a steady state of the patient and be used to determine increased edema with additional images (e.g., one or more images 83) captured over time. For example, patient 4, caregiver, or clinician may press external device 12 to a patient's neck, ankle, or hand, with a camera sensor and a flash facing the patient. In some examples, a device may be placed on external device 12 and patient 4 to temporarily fix external device 12 to patient 4 during the capture of one or more images 83.

For example, patient 4, a caregiver, or a clinician may activate flash 81 and record video which may be used to capture the variation in light reflected from the tissue of patient 4. For example, the light from flash 79 may travel through the tissue of patient 4. If more liquid is present in the tissue, more light is reflected and captured by camera sensor 79. If less liquid is present in the tissue, less light is reflected and captured by camera sensor 79.

Processing circuitry 80 may re-sample the captured video at a lower frequency to create an average signal. this signal may be further averaged by being added to a trend of past signals which may be stored in one or more images 83. Processing circuitry 80 may determine results by determining a difference between the average and a baseline, by determining a difference between the new measurement and the baseline, by determining a difference between the new measurement and a previous measurement, and/or by determining the amount of edema in a past predetermined number of days above the baseline.

Processing circuitry 80 may generate an indication for output regarding the results. The indication may include the results and may be sent to patient 4 to communicate “good” or “poor” results and/or to encourage the patient to take their fluid medication. Processing circuitry 80 may record the results and provide the indication to the clinician as well. In some examples, the results may include alphabetic rankings, numerical rankings, color coded rankings (e.g., green, yellow, or red), or the like.

Alternatively, or additionally, processing circuitry 80 may execute a heart failure algorithm an include the results with data from IMD 10 to determine heart failure score 89 which may have more specificity than it would have without result(s) 85. Such heart failure algorithm may provide a prediction of a heart failure event and/or a prediction of recovery from a heart failure event.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., data corresponding to one or more of a cardiac flow rate, optical sensor signals, an accelerometer signal, heart failure score 89, or other collected data) to external device 12. In turn, external device 12 may receive the collected data from IMD 10 and store the collected data in storage device 84. Additionally, or alternatively, processing circuitry 80 may export instructions to IMD 10 requesting IMD 10 to update one or more operational parameters of IMD 10.

A user, such as a clinician, patient 4, or a caregiver, may interact with external device 12 through user interface 86. User interface 86 includes a display (not shown), such as an LCD or LED display or other type of screen, with which processing circuitry 80 may present information related to IMD 10 (e.g., sensed physiological). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to patient 4, receiving voice commands from patient 4, or both. Storage device 84 may include instructions for operating user interface 86 and for managing power source 88.

Power source 88 is configured to deliver operating power to the components of external device 12. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate.

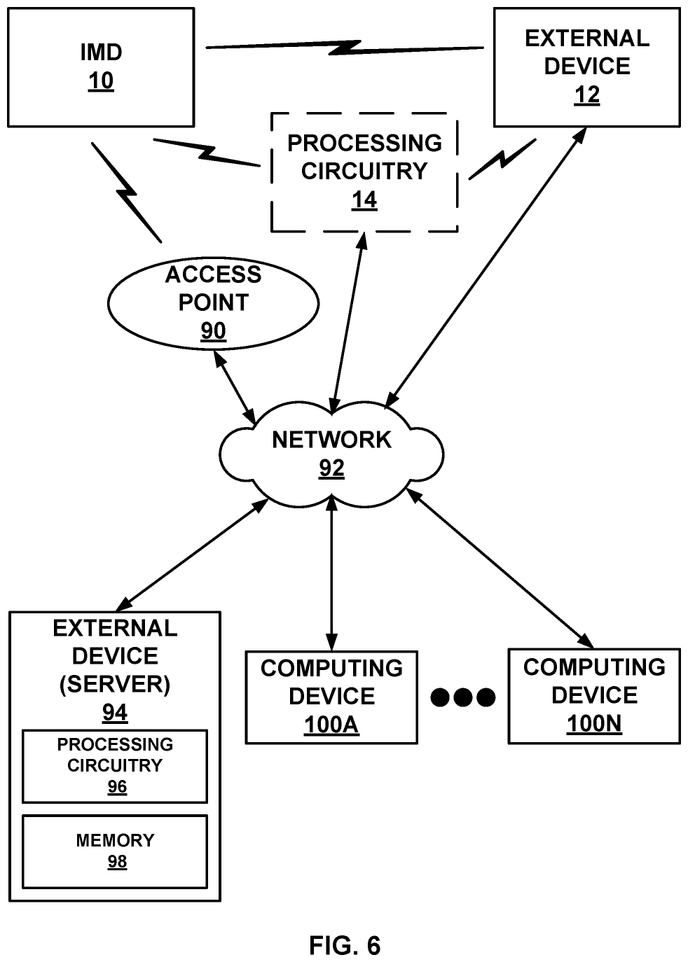

FIG. 6 is a block diagram illustrating an example system that includes an access point 90, a network 92, external computing devices, such as a server 94, and one or more other computing devices 100A-100N, which may be coupled to IMD 10, external device 12, and processing circuitry 14 via network 92, in accordance with one or more techniques described herein. In this example, IMD 10 may use communication circuitry 54 to communicate with external device 12 via a first wireless connection, and to communication with an access point 90 via a second wireless connection. In the example of FIG. 6, access point 90, external device 12, server 94, and computing devices 100A-100N are interconnected and may communicate with each other through network 92.

Access point 90 may include a device that connects to network 92 via any of a variety of connections, such as telephone dial-up, digital subscriber line (DSL), fiber optic, or cable modem connections. In other examples, access point 90 may be coupled to network 92 through different forms of connections, including wired or wireless connections. In some examples, access point 90 may be a user device, such as a tablet or smartphone, that may be co-located with the patient. In some examples, external device 12 and access point 90 may be same user computing device, e.g., of the patient, such as a tablet or smartphone. As discussed above, IMD 10 may be configured to transmit data, such a trigger to begin collecting one or more images 83, a heart failure score, optical sensor signals, an accelerometer signal, or other data collected by IMD 10 to external device 12. In addition, access point 90 may interrogate IMD 10, such as periodically or in response to a command from the patient or network 92, in order to retrieve parameter values determined by processing circuitry 50 of IMD 10, or other operational or patient data from IMD 10. Access point 90 may then communicate the retrieved data to server 94 via network 92.

In some cases, server 94 may be configured to provide a secure storage site for data that has been collected from IMD 10, and/or external device 12, such as one or more images 83, result(s) 85, one or more baseline images 87, and/or physiological parameters of patient 4. In some cases, server 94 may assemble data in web pages or other documents for viewing by trained professionals, such as clinicians, via computing devices 100A-100N. One or more aspects of the illustrated system of FIG. 6 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein.

Server 94 may include memory 98. Memory 98 includes computer-readable instructions that, when executed by processing circuitry 96, cause IMD 10 and processing circuitry 96 to perform various functions attributed to IMD 10 and processing circuitry 96 herein. Memory 98 may include any volatile, non-volatile, magnetic, optical, or electrical media, such as RAM, ROM, NVRAM, EEPROM, flash memory, or any other digital media. In some examples, memory 98 may include artificial intelligence model 78 (not shown in FIG. 6) and processing circuitry 96 may execute artificial intelligence model 78 to generate result(s) 85.

In some examples, one or more of computing devices 100A-100N (e.g., device 100A) may be a tablet or other smart device located with a clinician, by which the clinician may program, receive alerts from, and/or interrogate IMD 10. For example, the clinician may access data corresponding to physiological parameters of patient 4 determined by IMD 10, external device 12, processing circuitry 14, and/or server 94 through device 100A, such as when patient 4 is in between clinician visits, to check on a status of a medical condition, such as heart failure. In some examples, the clinician may enter instructions for a medical intervention for patient 4 into an app in device 100A, such as based on a status of a patient condition determined by IMD 10, external device 12, processing circuitry 14, or any combination thereof, or based on other patient data known to the clinician. Device 100A then may transmit the instructions for medical intervention to another of computing devices 100A-100N (e.g., device 100B) located with patient 4 or a caregiver of patient 4. For example, such instructions for medical intervention may include an instruction to change a drug dosage, timing, or selection, to schedule a visit with the clinician, to take their fluid medication, or to seek medical attention. In further examples, device 100B may generate an indication for output, such as an alert to patient 4 based on a status of a medical condition of patient 4 determined by IMD 10, which may enable patient 4 proactively to seek medical attention prior to receiving instructions for a medical intervention. In this manner, patient 4 may be empowered to take action, as needed, to address their medical status, which may help improve clinical outcomes for patient 4.

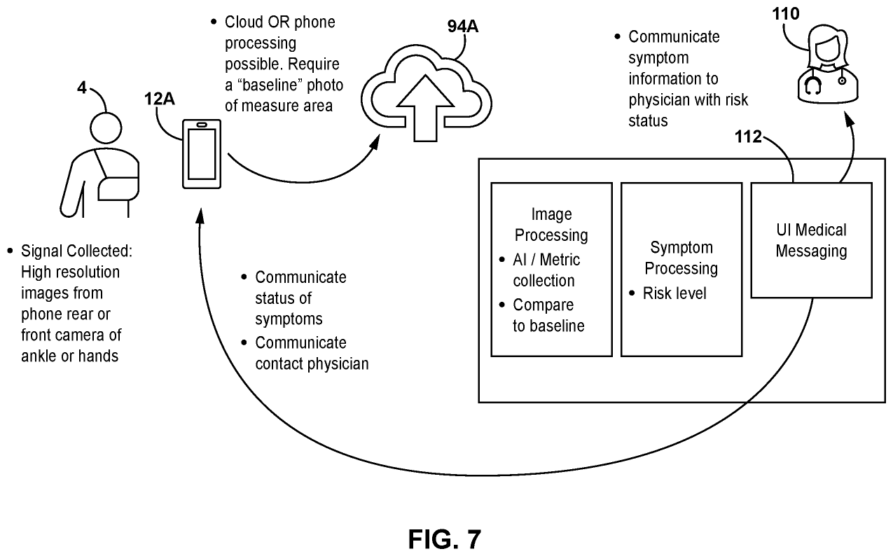

FIG. 7 is a conceptual diagram illustrating the use of photography to determine an edema result. In the example of FIG. 7, cellular phone 12A may be an example of external device 12 of FIGS. 1 and 5, and cloud computing environment 94A may be an example of server 94 of FIG. 6. Patient 4 may collect one or more images 83, which may be high resolution images from a camera sensor of cellular phone 12A. For example, the one or more images may be of a region of interest on an ankle of patient 4 or a hand of patient 4, or other anatomy described herein. In some examples, cellular phone 12A may process one or more images 83 to result(s) 85. In some examples, cloud computing environment 94A may process one or more images 83 to result(s) 85. In some examples, either cellular phone 12A, cloud computing environment 94A, or a combination thereof, may execute artificial intelligence model 78 when processing one or more images 83 to result(s) 85. Either cellular phone 12A or cloud computing environment 94A may compare one or more images 83 to one or more baseline images 87 and determine result(s) 85 based on the comparison.

Either cellular phone 12A or cloud computing environment 94A may communicate status of symptoms of patient 4. For example, either cellular phone 12A or cloud computing environment 94A may generate an indication for output and transmit the indication to patient 4, a caregiver, clinician 110, or any combination thereof. Such an indication may be displayed to patient 4 or a caregiver via cellular phone 12A or to clinician 110 via medical messaging user interface 112, which, for example, may be part of computing device 100A of FIG. 6. In some examples, the indication may include one or more images 83, result(s), heart failure score 89 (which, in some examples, may be based on both sensor signals from sensors of IMD 10 and the determined result), etc.

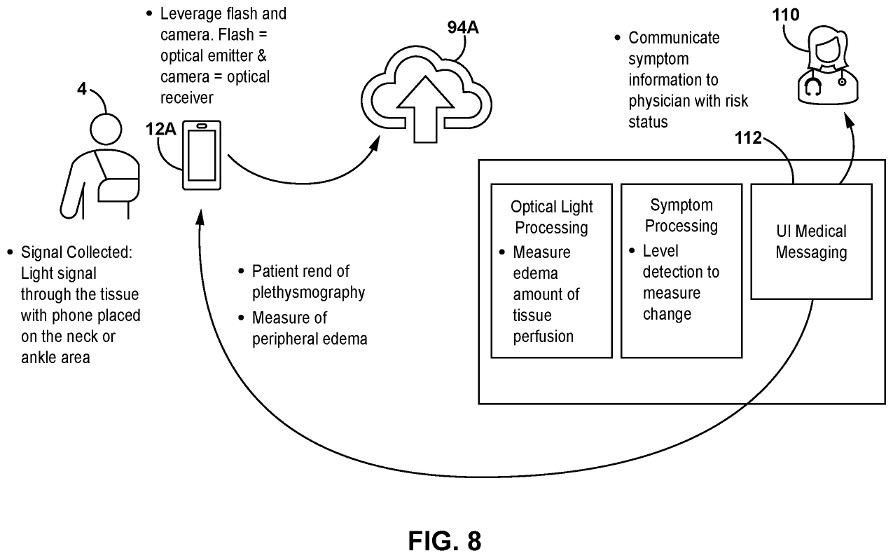

FIG. 8 is a conceptual diagram illustrating the use of photography to determine an oxygen saturation or perfusion result. In the example of FIG. 8, cellular phone 12A may be an example of external device 12 of FIGS. 1 and 5, and cloud computing environment 94A may be an example of server 94 of FIG. 6. Patent 4 may initiate a flash on cellular phone 12A and may collect one or more images 83 from a camera sensor of cellular phone 12A. In this example, one or more images 83 may include a video. For example, the camera sensor may capture light from the flash that is reflected off of tissue of patient 4. For example, one or more images 83 may be of a region of interest on a neck of patient 4 or an ankle of patient 4, or other anatomy described herein. In this example patient 4 may press cellular phone 12A against the region of interest when capturing one or more images 83. In some examples, cellular phone 12A may process one or more images 83 to result(s) 85. In some examples, cloud computing environment 94A may process one or more images 83 to generate result(s) 85. In some examples, either cellular phone 12A, cloud computing environment 94A, or a combination thereof, may execute artificial intelligence model 78 when processing one or more images 83 to generate result(s) 85. Either cellular phone 12A or cloud computing environment 94A may compare one or more images 83 to one or more baseline images 87 and determine result(s) 85 based on the comparison. Such a result may be a measure of peripheral edema or tissue perfusion.

Either cellular phone 12A or cloud computing environment 94A may generate an indication for output and transmit the indication to patient 4, a caregiver, clinician 110, or any combination thereof. Such an indication may be displayed to patient 4 or a caregiver via cellular phone 12A or to clinician 110 via medical messaging user interface 112, which, for example, may be part of computing device 100A of FIG. 6. In some examples, the indication may include, one or more images 83, result(s) 85, heart failure score 89 (which, in some examples, may be based on both sensor signals from sensors of IMD 10 and the determined result), etc.

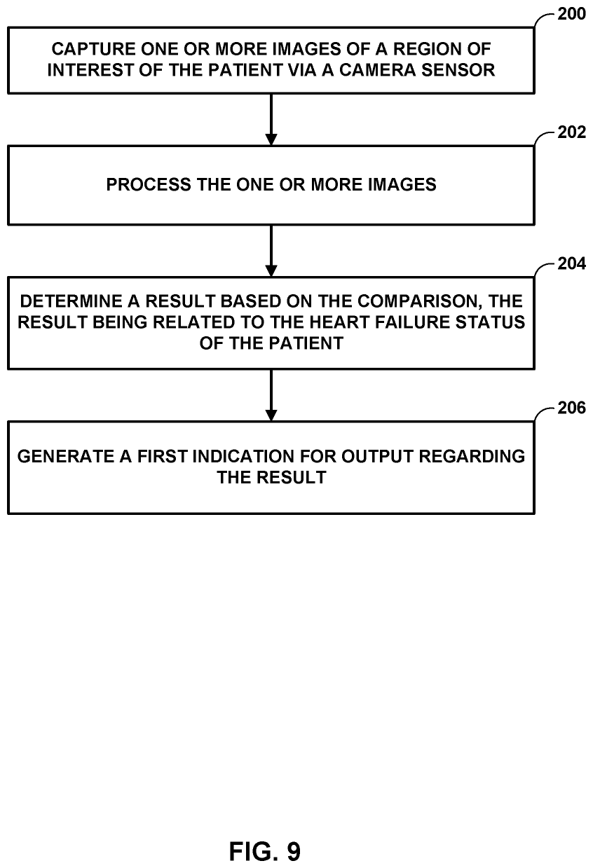

FIG. 9 is a flow diagram illustrating an example of biochemical sensor or bacterial sensor deployment techniques according to one or more aspects of this disclosure. The example of FIG. 9 is discussed with respect to external device 12 of FIG. 5, but these techniques may be implemented in any combination of devices of system 2 (FIG. 1) or of FIGS. 6-8.

Processing circuitry 50 may capture one or more images 83 of a region of interest of patient 4 via camera sensor 79 (200). For example, processing circuitry 50 may determine that patient 4 has pressed a camera shutter button and control camera sensor 79 to capture one or more images 83 of the region of interest of patient 4. Processing circuitry 80 may process one or more images 83 (202). For example, processing circuitry 80 may compare one or more images 83 to one or more baseline images 87. In some examples, one or more baseline images 87 include one or more previous images captured by a clinician via a cellular phone, a table computer, or a digital camera. In some examples, processing circuitry 80 may apply artificial intelligence model 78 when processing one or more images 83. In some examples, processing circuitry 80 may determine at least one of: eye color; glassiness; changes from one or more reference images, the changes from one or more reference images comprising at least one of changes in color of a sclera or iris, changes in clarity of an eye lens, changes in glassiness, changes in presence or prominence of vasculature, changes in evidence of emboli, changes in color of a tear duct; or changes between sequential captured images, the changes between sequential capture images comprising at least one of changes in a pupil response to a flash, or changes in an eye tracking a moving image on a screen.

For example, processing circuitry 80 may determining at least one of: a change in size of tissue in the region of interest between the at least one image of the region of interest while the user is applying pressure to the region of interest and the at least one image of the region of interest after the user releases the pressure from the region of interest; a change in color of the tissue in the region of interest between the at least one image of the region of interest while the user is applying pressure to the region of interest and the at least one image of the region of interest after the user releases the pressure from the region of interest; a rate of change in the color of the tissue in the region of interest between the at least one image of the region of interest while the user is applying pressure to the region of interest and the at least one image of the region of interest after the user releases the pressure from the region of interest, or a rate of change in a slope of a skin surface of the tissue in the region of interest between the at least one image of the region of interest while the user is applying pressure to the region of interest and the at least one image of the region of interest after the user releases the pressure from the region of interest.

Processing circuitry 80 may determine a result based on the processing, the result being related to the heart failure status of the patient (204). For example, processing circuitry 80 may determine a measure of edema, a measure of tissue oxygenation, or a measure of perfusion based on the processing.

Processing circuitry 80 may generate a first indication for output regarding the result (206). For example, the first indication may include at least one of a ranking on a heart failure status scale or one or more images 83. In some examples, processing circuitry 80 may output the first indication to one or more users. In some examples, the one or more users comprise at least one of clinician 110 (FIGS. 7-8), patient 4, or a caregiver.

In some examples, processing circuitry 80 may receive a trigger from IMD 10 and based on receiving the trigger from IMD 10, output instructions via user interface 86 to a user, the instructions including instructions to take one or more images 83 using camera sensor 79 of the region of interest. In some examples, the instructions further include a map of the region of interest. In some examples, processing circuitry 80 may determine a heart failure score 89 based on sensor signals from IMD 10 and the result and output heart failure score 89 to one or more users. In some examples, the trigger is based on one or more sensor signals from one or more sensors of the IMD 10. In some examples, the one or more sensors comprise a respiration sensor. In some examples, processing circuitry 80 may tune at least one of the one or more sensors based on the result. For example, the sensor signals from IMD 10 may provide different indications than the result and processing circuitry 80 may adjust at least one of the one or more sensors to compensate for the different indications.

In some examples, the region of interest includes an ankle of patient 4, a hand of patient 4, a leg of patient 4, a foot of patient 4, a face of patient 4, a neck of patient 4, an eye of patient 4, or an eyelid of patient 4. In some examples, processing circuitry 80 may monitor the use of medication of the patient, wherein the use of medication comprises at least one of a type of the medication, a time of taking of the medication, an amount taken of the medication, and wherein the result is further based on the use of medication of the patient.

In some examples, one or more images 83 comprise at least one image of the region of interest while the user is applying pressure to the region of interest, and at least one image of the region of interest after the user releases the pressure from the region of interest. In some examples, the user applies the pressure via a finger or via constriction from clothing. In some examples,

In some examples, one or more images 83 comprise a video of light from flash 81 reflected back to camera sensor 79 when at least one of flash 81 or camera sensor 79 is in contact with the region of interest. In some examples, processing one or more images 83 includes resampling the video at a lower frequency than the video to generate an average.

In some examples, processing circuitry 80 may determine that the result does not meet a threshold (e.g., is ranked as poor) and periodically repeat capturing one or more images 83 of the region of interest of the patient via camera sensor 79, processing one or more images 83, and generating the result based on the processing, until the result does meet the threshold (e.g., is ranked as good). In some examples, one or more images 83 are captured via a cellular phone, a tablet computer, or a digital camera.

In some examples, the result comprises a prediction of worsening heart failure. In some examples, processing circuitry 80 is further configured to output instructions to a user for the user to use the camera sensor to capture the one or more images. In some examples, processing circuitry 80 is further configured to provide feedback to the user on the positioning of the camera sensor. In some examples, as part of providing feedback, processing circuitry 80 is configured to determine a landmark (e.g., an ankle, a thumb, a big toe, etc.) in anatomy of a patient relative to the area of interest. For example, processing circuitry 80 may use artificial intelligence, such as computer vision, to identify a landmark. In some examples, the instructions include instructions for the user to use the camera sensor to capture a plurality of images from a plurality of angles, and wherein the processing circuitry is further configured to stitch at least two of the plurality of images together to generate a composite image. In some examples, processing circuitry 80 is further configured to determine that the user has not yet used the camera sensor to capture the one or more images, generate a reminder for the user to use the camera sensor to capture the one or more images, and output the reminder for the user to use the camera sensor to capture the one or more images. For example, the reminder may be an audible reminder, a haptic reminder (e.g., vibration), and/or a visual reminder. In some examples, processing circuitry 80 is further configured to repetitively and at a more frequent occurrence, output the reminder for the user to use the camera sensor to capture the one or more images until the user uses the camera sensor to capture the one or more images.

In some examples, the region of interest comprises a first region of interest, wherein the one or more images comprise a first one or more images. In some examples, processing circuitry is further configured to capture a second one or more images from a second region of interest of the patient within a predetermined timeframe (e.g., within 10 minutes) of the capture of the first one or more images of the first region of interest, the second region of interest being in a different region of an anatomy of the patient than the first region of interest. For example, the second region of interest may be located more than a distance, such as 12 inches, from the first region of interest. In such examples, processing circuitry 80 is configured to process the second one more images, and determine the result based on both the processing of the first one or more images and the processing of the second one or more images.

In some examples, the one or more images comprise a first one or more images, wherein the result is a first result. In some examples, processing circuitry 80 is further configured to store the first one or more images in memory and capture a second one or more images of the region of interest of the patient via the camera sensor after a time period. In such examples, processing circuitry 80 is configured to compare the second one or more images to the first one or more images. In such examples, processing circuitry 80 is configured to determine a second result based on the comparison, the second result being related to the heart failure status of the patient and generate a second indication for output regarding the second result.

In some examples, the one or more images comprise a first one or more images, wherein the result is a first result, and processing circuitry 80 is further configured to store the first one or more images in memory and compare the first one or more images to a second one or more images, the second one or more images being one or more images captured of the region of interest of a cohort. For example, the cohort may be similarly situated medically to patient 4. In such examples, processing circuitry 80 may be further configured to determine a second result based on the comparison, the second result being related to the heart failure status of the patient, and generate a second indication for output regarding the second result.

In some examples, processing circuitry 80 is further configured to output the first indication to a clinician user interface, and wherein the first indication comprises at least one of the one or more images, one or more heart failure indicators, or one or more physiological parameters from sensor signals of an implantable medical device. In some examples, processing circuitry 80 is further configured to output the first indication to a patient user interface contemporaneously with outputting the first indication to the clinician user interface.

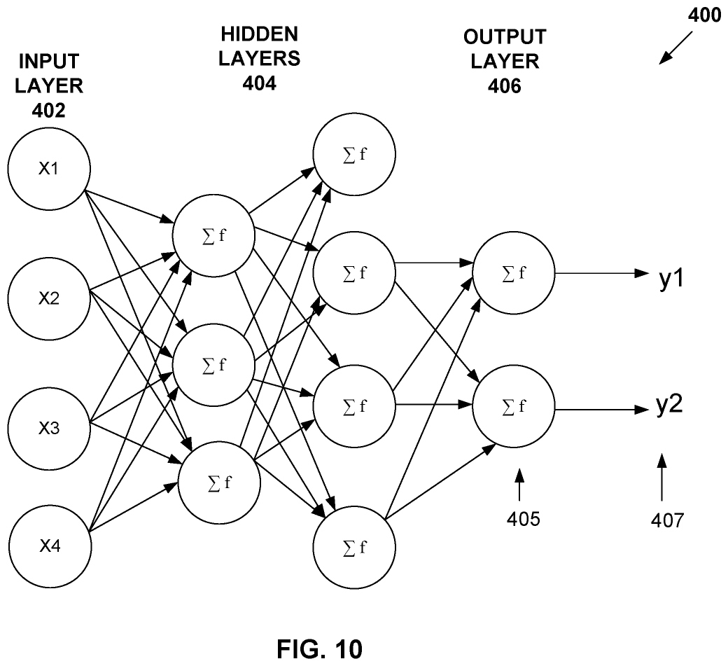

FIG. 10 is a conceptual diagram illustrating an example machine learning model 400 configured to determine a heart failure state or other health condition state based on comparison of images and, in some cases, physiological parameter values, e.g., sensed by IMD 10. Machine learning model 400 is an example of a deep learning model, or deep learning algorithm. One or more of IMD 10, external device 12, or server 94 may train, store, and/or utilize machine learning model 400, but other devices may apply inputs associated with a particular patient to machine learning model 400 in other examples. Some non-limiting examples of machine learning techniques include Bayesian probability models, Support Vector Machines, K-Nearest Neighbor algorithms, and Multi-layer Perceptron.

As shown in the example of FIG. 10, machine learning model 400 may include three layers. These three layers include input layer 402, hidden layer 404, and output layer 406. Output layer 406 comprises the output from the transfer function 405 of output layer 406. Input layer 402 represents each of the input values X1 through X4 provided to machine learning model 400. The number of inputs may be less than or greater than 4, including much greater than 4, e.g., hundreds or thousands. In some examples, the input values may be any of the of values described above. In some examples, input values may include parameter values described herein, e.g., images or values representing comparison of images, and other physiological parameter values.

Each of the input values for each node in the input layer 402 is provided to each node of hidden layer 404. In the example of FIG. 10, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between the inputs determining a score indicative of whether a set of inputs may be representative of a particular risk level or health state. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be or a score indicative of a risk level (e.g., likelihood or probability) of an adverse health event due to a dialysis session. By applying the patient parameter data to a machine learning model, such as machine learning model 400, processing circuitry of system 2 is able to determine health conditions, e.g. heart failure risk, with great accuracy, specificity, and sensitivity. This may facilitate improved therapy efficacy and safety, and improved patient health.

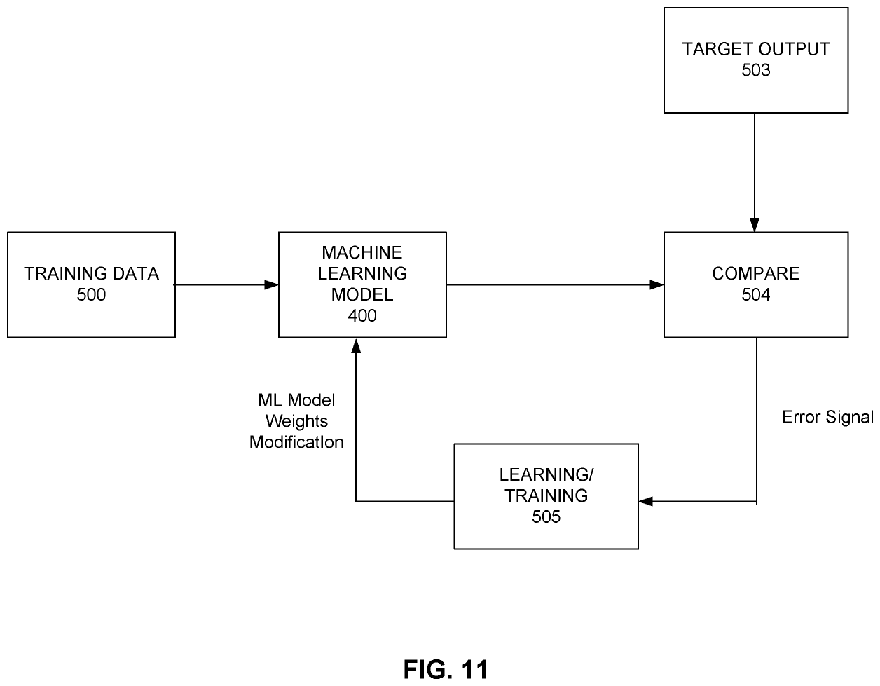

FIG. 11 is an example of a machine learning model 400 being trained using supervised and/or reinforcement learning techniques. Machine learning model 400 may be implemented using any number of models for supervised and/or reinforcement learning, such as but not limited to, an artificial neural network, a decision tree, naïve Bayes network, support vector machine, or k-nearest neighbor model, to name only a few examples. In some examples, processing circuitry one or more of IMD 10, external device 12, and/or server 94 initially trains the machine learning model 400 based on training set data 500 including numerous instances of input data corresponding to various risk levels and/or other patient conditions. An output of the machine learning model 400 may be compared 504 to the target output 503, e.g., as determined based on the label. Based on an error signal representing the comparison, the processing circuitry implementing a learning/training function 505 may send or apply a modification to weights of machine learning model 400 or otherwise modify/update the machine learning model 400. For example, one or more of IMD 10, computing device 6, external device 12, and/or computing system 20 may, for each training instance in the training set 500, modify machine learning model 400 to change a score generated by the machine learning model 400 in response to data applied to the machine learning model 400.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, FRAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

This disclosure includes the following non-limiting examples.

Example 1. A system for monitoring a heart failure status of a patient, the system comprising: a memory configured to store one or more images; a camera sensor; and
processing circuitry communicatively coupled to the memory, and the camera sensor, the processing circuitry being configured to: capture one or more images of a region of interest of the patient via the camera sensor; process the one or more images; determine a result based on the processing, the result being related to the heart failure status of the patient; and generate a first indication for output regarding the result.

Example 2. The system of claim 1, wherein the first indication comprises at least one of a ranking on a heart failure status scale or the one or more images, and wherein the processing circuitry is further configured to output the first indication to one or more users.

Example 3. The system of claim 2, wherein the one or more users comprise at least one of a clinician, the patient, or a caregiver.

Example 4. The system of any of claims 1-3, wherein the processing circuitry is further configured to: receive a trigger from an implantable medical device; and
based on receiving the trigger from the implantable medical device, output instructions via a user interface to a user, the instructions comprising instructions to take the one or more images using the camera sensor of the region of interest.

Example 5. The system of claim 4, wherein the instructions further comprise a map of the region of interest.

Example 6. The system of claim 4 or claim 5, wherein the processing circuitry is further configured to: determine a heart failure score based on sensor signals from the implantable medical device and the result; and output the heart failure score to one or more users.

Example 7. The system of any of claims 4-6, wherein the trigger is based on one or more sensor signals from one or more sensors of the implantable medical device.

Example 8. The system of claim 7, wherein the one or more sensors comprise a respiration sensor.

Example 9. The system of claim 7 or claim 8, wherein the processing circuitry is further configured to tune at least one of the one or more sensors based on the result.

Example 10. The system of any of claims 1-9, wherein the region of interest comprises: an ankle of the patient; a hand of the patient; a leg of the patient; a foot of the patient; a face of the patient; a neck of the patient; an eye of the patient; or an eyelid of the patient.

Example 11. The system of any of claims 1-10, wherein the processing circuitry is further configured to monitor the use of medication of the patient, wherein the use of medication comprises at least one of a type of the medication, a time of taking of the medication, an amount taken of the medication, and wherein the result is further based on the use of medication of the patient.

Example 12. The system of any of claims 1-11, wherein as part of processing the one or more images, the processing circuitry is configured to compare the one or more images to one or more baseline images, wherein the one or more baseline images comprise one or more previous images captured by a clinician via a cellular phone, a tablet computer, or a digital camera.

Example 13. The system of claim 12, wherein as part of processing the one or more images, the processing circuitry is configured to apply an artificial intelligence model to the one or more images.

Example 14. The system of any of claims 1-13, wherein as part of processing the one or more images, the processing circuitry is configured to determine at least one of: eye color; glassiness; changes from one or more reference images, the changes from one or more reference images comprising at least one of changes in color of a sclera or iris, changes in clarity of an eye lens, changes in glassiness, changes in presence or prominence of vasculature, changes in evidence of emboli, changes in color of a tear duct; or
changes between sequential captured images, the changes between sequential capture images comprising at least one of changes in a pupil response to a flash, or changes in an eye tracking a moving image on a screen.

Example 15. The system of any of claims 1-14, wherein the one or more images comprise: at least one image of the region of interest while the user is applying pressure to the region of interest; and at least one image of the region of interest after the user releases the pressure from the region of interest.

Example 16. The system of claim 15, wherein the user applies the pressure via a finger or via constriction from clothing.

Example 17. The system of claim 15 or claim 16, wherein as part of processing the one or more images, the processing circuitry is configured to determine at least one of: a change in size of tissue in the region of interest between the at least one image of the region of interest while the user is applying pressure to the region of interest and the at least one image of the region of interest after the user releases the pressure from the region of interest; a change in color of the tissue in the region of interest between the at least one image of the region of interest while the user is applying pressure to the region of interest and the at least one image of the region of interest after the user releases the pressure from the region of interest; a rate of change in the color of the tissue in the region of interest between the at least one image of the region of interest while the user is applying pressure to the region of interest and the at least one image of the region of interest after the user releases the pressure from the region of interest; or a rate of change in a slope of a skin surface of the tissue in the region of interest between the at least one image of the region of interest while the user is applying pressure to the region of interest and the at least one image of the region of interest after the user releases the pressure from the region of interest.

Example 18. The system of any of claims 1-14, wherein the system further comprises a flash, and wherein the one or more images comprise a video of light from the flash reflected back to the camera sensor when at least one of the flash or the camera sensor is in contact with the region of interest.

Example 19. The system of claim 18, wherein as part of processing the one or more images, the processing circuitry is configured to resample the video at a lower frequency than the video to generate an average.

Example 20. The system of any of claims 1-19, wherein the processing circuitry is configured to: determine that the result does not meet a threshold; and periodically repeat capturing one or more images of the region of interest of the patient via the camera sensor, processing the one or more images, and generating the result based on the processing, until the result meets the threshold.

Example 21. The system of any of claims 1-20, wherein the system comprises a cellular phone, a tablet computer, or a digital camera.

Example 22. The system of any of claims 1-21, wherein the result comprises a prediction of worsening heart failure.

Example 23. The system of any of claims 1-22, wherein the processing circuitry is further configured to output instructions to a user for the user to use the camera sensor to capture the one or more images.

Example 24. The system of claim 23, wherein the processing circuitry is further configured to provide feedback to the user on the positioning of the camera sensor.

Example 25. The system of claim 24, wherein as part of providing feedback, the processing circuitry is configured to determine a landmark in anatomy of a patient relative to the area of interest.

Example 26. The system of any of claims 23-25, wherein the instructions comprise instructions for the user to use the camera sensor to capture a plurality of images from a plurality of angles, and wherein the processing circuitry is further configured to stitch at least two of the plurality of images together to generate a composite image.

Example 27. The system of any of claims 23-26, wherein the processing circuitry is further configured to: determine that the user has not yet used the camera sensor to capture the one or more images; generate a reminder for the user to use the camera sensor to capture the one or more images; and output the reminder for the user to use the camera sensor to capture the one or more images.

Example 28. The system of claim 27, wherein the processing circuitry is further configured to repetitively and at a more frequent occurrence, output the reminder for the user to use the camera sensor to capture the one or more images until the user uses the camera sensor to capture the one or more images.

Example 29. The system of any of claims 1-28, wherein the region of interest comprises a first region of interest, wherein the one or more images comprise a first one or more images, and wherein the processing circuitry is further configured to: capture a second one or more images from a second region of interest of the patient within a predetermined timeframe of the capture of the first one or more images of the first region of interest, the second region of interest being in a different region of an anatomy of the patient than the first region of interest; process the second one more images; and determine the result based on both the processing of the first one or more images and the processing of the second one or more images.

Example 30. The system of any of claims 1-28, wherein the one or more images comprise a first one or more images, wherein the result is a first result, and wherein the processing circuitry is further configured to: store the first one or more images in memory; capture a second one or more images of the region of interest of the patient via the camera sensor after a time period; compare the second one or more images to the first one or more images; determine a second result based on the comparison, the second result being related to the heart failure status of the patient; and generate a second indication for output regarding the second result.

Example 31. The system of any of claims 1-28, wherein the one or more images comprise a first one or more images, wherein the result is a first result, and wherein the processing circuitry is further configured to: store the first one or more images in memory; compare the first one or more images to a second one or more images, the second one or more images being one or more images captured of the region of interest of a cohort; determine a second result based on the comparison, the second result being related to the heart failure status of the patient; and generate a second indication for output regarding the second result.

Example 32. The system of any of claims 1-31, wherein the processing circuitry is further configured to output the first indication to a clinician user interface, and wherein the first indication comprises at least one of the one or more images, one or more heart failure indicators, or one or more physiological parameters from sensor signals of an implantable medical device.

Example 33. The system of claim 32, wherein the processing circuitry is further configured to output the first indication to a patient user interface contemporaneously with outputting the first indication to the clinician user interface.

Example 34. A method of monitoring a heart failure status of a patient, the method comprising: capturing, by processing circuitry, one or more images of a region of interest of the patient via a camera sensor; processing, by the processing circuitry, the one or more images; determining, by the processing circuitry, a result based on the processing, the result being related to the heart failure status of the patient; and generating, by the processing circuitry, a first indication for output regarding the result.

Example 35. The method of claim 34, wherein the first indication comprises at least one of a ranking on a heart failure status scale or the one or more images, the method further comprising outputting the first indication to one or more users.

Example 36. The method of claim 35, wherein the one or more users comprise at least one of a clinician, the patient, or a caregiver.

Example 37. The method of any of claims 34-36, further comprising: receiving, by the processing circuitry, a trigger from an implantable medical device; and based on receiving the trigger from the implantable medical device, outputting, by the processing circuitry, instructions via a user interface to a user, the instructions comprising instructions to take the one or more images using the camera sensor of the region of interest.

Example 38. The method of claim 37, wherein the instructions further comprise a map of the region of interest.

Example 39. The method of claim 37 or claim 38, further comprising: determining, by the processing circuitry, a heart failure score based on sensor signals from the implantable medical device and the result; and outputting, by the processing circuitry, the heart failure score to one or more users.

Example 40. The method of any of claims 37-39, wherein the trigger is based on one or more sensor signals from one or more sensors of the implantable medical device.

Example 41. The method of claim 40, wherein the one or more sensors comprise a respiration sensor.

Example 42. The method of claim 40 or claim 41, further comprising tuning, by the processing circuitry, at least one of the one or more sensors based on the result.

Example 43. The method of any of claims 34-42, wherein the region of interest comprises: an ankle of the patient; a hand of the patient; a leg of the patient; a foot of the patient; a face of the patient; a neck of the patient; an eye of the patient; or an eyelid of the patient.

Example 44. The method of any of claims 34-43, further comprising monitoring, by the processing circuitry, the use of medication of the patient, wherein the use of medication comprises at least one of a type of the medication, a time of taking of the medication, an amount taken of the medication, and wherein the result is further based on the use of medication of the patient.

Example 45. The method of any of claims 34-44, wherein processing the one or more images comprises comparing the one or more images to one or more baseline images, wherein the one or more baseline images comprise one or more previous images captured by a clinician via a cellular phone, a tablet computer, or a digital camera.

Example 46. The method of claim 45, wherein processing the one or more images comprises applying an artificial intelligence model to the one or more images.

Example 47. The method of any of claims 34-46, wherein processing the one or more images, comprises determining at least one of: eye color; glassiness; changes from one or more reference images, the changes from one or more reference images comprising at least one of changes in color of a sclera or iris, changes in clarity of an eye lens, changes in glassiness, changes in presence or prominence of vasculature, changes in evidence of emboli, changes in color of a tear duct; or changes between sequential captured images, the changes between sequential capture images comprising at least one of changes in a pupil response to a flash, or changes in an eye tracking a moving image on a screen.

Example 48. The method of any of claims 34-47, wherein the one or more images comprise: at least one image of the region of interest while the user is applying pressure to the region of interest; and at least one image of the region of interest after the user releases the pressure from the region of interest.

Example 49. The method of claim 48, wherein the user applies the pressure via a finger or via constriction from clothing.

Example 50. The method of claim 47 or claim 48, wherein processing the one or more images comprises determining at least one of: a change in size of tissue in the region of interest between the at least one image of the region of interest while the user is applying pressure to the region of interest and the at least one image of the region of interest after the user releases the pressure from the region of interest; a change in color of the tissue in the region of interest between the at least one image of the region of interest while the user is applying pressure to the region of interest and the at least one image of the region of interest after the user releases the pressure from the region of interest; a rate of change in the color of the tissue in the region of interest between the at least one image of the region of interest while the user is applying pressure to the region of interest and the at least one image of the region of interest after the user releases the pressure from the region of interest; or a rate of change in a slope of a skin surface of the tissue in the region of interest between the at least one image of the region of interest while the user is applying pressure to the region of interest and the at least one image of the region of interest after the user releases the pressure from the region of interest.

Example 51. The method of any of claims 34-50, wherein the one or more images comprise a video of light from a flash reflected back to the camera sensor when at least one of the flash or the camera sensor is in contact with the region of interest.

Example 52. The method of claim 51, wherein processing the one or more images comprises resampling the video at a lower frequency than the video to generate an average.

Example 53. The method of any of claims 34-52, further comprising: determining, by the processing circuitry, that the result does not meet a threshold; and periodically repeating capturing one or more images of the region of interest of the patient via the camera sensor, processing the one or more images, and generating the result based on the processing, until the result does meet the threshold.

Example 54. The method of any of claims 34-53, wherein the one or more images are captured via a cellular phone, a tablet computer, or a digital camera.

Example 55. The method of any of claims 34-54, wherein the result comprises a prediction of worsening heart failure.

Example 56. The method of any of claims 34-55, further comprising outputting, by the processing circuitry, instructions to a user for the user to use the camera sensor to capture the one or more images.

Example 57. The method of claim 56, further comprising, providing, by the processing circuitry, feedback to the user on the positioning of the camera sensor.

Example 58. The method of claim 57, wherein providing feedback comprises, determining a landmark in anatomy of a patient relative to the area of interest.

Example 59. The method of any of claims 56-58, wherein the instructions comprise instructions for the user to use the camera sensor to capture a plurality of images from a plurality of angles, and wherein the method further comprises stitching, by the processing circuitry, at least two of the plurality of images together to generate a composite image.

Example 60. The method of any of claims 56-59, further comprising: determining, by the processing circuitry, that the user has not yet used the camera sensor to capture the one or more images; generating, by the processing circuitry, a reminder for the user to use the camera sensor to capture the one or more images; and outputting, by the processing circuitry, the reminder for the user to use the camera sensor to capture the one or more images.

Example 61. The method of claim 60, further comprising repetitively and at a more frequent occurrence, outputting, by the processing circuitry, the reminder for the user to use the camera sensor to capture the one or more images until the user uses the camera sensor to capture the one or more images.

Example 62. The method of any of claims 34-61, wherein the region of interest comprises a first region of interest, wherein the one or more images comprise a first one or more images, and wherein the method further comprises: capturing, by the processing circuitry, a second one or more images from a second region of interest of the patient within a predetermined timeframe of the capture of the first one or more images of the first region of interest, the second region of interest being in a different region of an anatomy of the patient than the first region of interest; processing, by the processing circuitry, the second one more images; and determining, by the processing circuitry, the result based on both the processing of the first one or more images and the processing of the second one or more images.

Example 63. The method of any of claims 34-61, wherein the one or more images comprise a first one or more images, wherein the result is a first result, and wherein the method further comprises: storing, by the processing circuitry, the first one or more images in memory; capturing, by the processing circuitry, a second one or more images of the region of interest of the patient via the camera sensor after a time period; comparing, by the processing circuitry, the second one or more images to the first one or more images; determining, by the processing circuitry, a second result based on the comparison, the second result being related to the heart failure status of the patient; and generating, by the processing circuitry, a second indication for output regarding the second result.

Example 64. The method of any of claims 34-61, wherein the one or more images comprise a first one or more images, wherein the result is a first result, and wherein the method further comprises: storing, by the processing circuitry, the first one or more images in memory; comparing, by the processing circuitry, the first one or more images to a second one or more images, the second one or more images being one or more images captured of the region of interest of a cohort; determining, by the processing circuitry, a second result based on the comparison, the second result being related to the heart failure status of the patient; and generating, by the processing circuitry, a second indication for output regarding the second result.

Example 65. The method of any of claims 34-64, further comprising outputting, by the processing circuitry, the first indication to a clinician user interface, and wherein the first indication comprises at least one of the one or more images, one or more heart failure indicators, or one or more physiological parameters from sensor signals of an implantable medical device.

Example 66. The method of claim 65, further comprising outputting, by the processing circuitry, the first indication to a patient user interface contemporaneously with outputting the first indication to the clinician user interface.

Example 67. A non-transitory computer-readable storage medium storing instructions, which when executed, cause processing circuitry to: capture one or more images of a region of interest of interest patient via the camera sensor; process the one or more images; determine a result based on the processing, the result being related to the heart failure status of the patient; and generate a first indication for output regarding the result.

Various examples have been described. These and other examples are within the scope of the following claims.

1. A system for monitoring a heart failure status of a patient, the system comprising:
a memory configured to store one or more images;
a camera sensor; and
processing circuitry communicatively coupled to the memory, and the camera sensor, the processing circuitry being configured to:
capture one or more images of a region of interest of the patient via the camera sensor;
process the one or more images;
determine a result based on the processing, the result being related to the heart failure status of the patient; and
generate a first indication for output regarding the result.

2. The system of claim 1, wherein the first indication comprises at least one of a ranking on a heart failure status scale or the one or more images, and wherein the processing circuitry is further configured to output the first indication to one or more users.

3. The system of claim 2, wherein the one or more users comprise the patient.

4. The system of claim 1, wherein the processing circuitry is further configured to:
receive a trigger from an implantable medical device; and
based on receiving the trigger from the implantable medical device, output instructions via a user interface to a user, the instructions comprising instructions to take the one or more images using the camera sensor of the region of interest.

5. The system of claim 4, wherein the instructions further comprise a map of the region of interest.

6. The system of claim 4, wherein the processing circuitry is further configured to:
determine a heart failure score based on sensor signals from the implantable medical device and the result; and
output the heart failure score to one or more users.

7. The system of claim 4, wherein the trigger is based on one or more sensor signals from one or more sensors of the implantable medical device.

8. The system of claim 7, wherein the one or more sensors comprise a respiration sensor.

9. The system of claim 7, wherein the processing circuitry is further configured to tune at least one of the one or more sensors based on the result.

10. The system of claim 1, wherein the region of interest comprises:
an ankle of the patient;
a hand of the patient;
a leg of the patient;
a foot of the patient;
a face of the patient;
a neck of the patient;
an eye of the patient; or
an eyelid of the patient.

11. The system of claim 1, wherein the processing circuitry is further configured to monitor the a use of medication of the patient, wherein the use of medication comprises at least one of a type of the medication, a time of taking of the medication, an amount taken of the medication, and wherein the result is further based on the use of medication of the patient.

12. The system of claim 1, wherein as part of processing the one or more images, the processing circuitry is configured to compare the one or more images to one or more baseline images, wherein the one or more baseline images comprise one or more previous images captured by a clinician via a cellular phone, a tablet computer, or a digital camera.

13. The system of claim 12, wherein as part of processing the one or more images, the processing circuitry is configured to apply an artificial intelligence model to the one or more images.

14. The system of claim 1, wherein the one or more images comprise:
at least one image of the region of interest while the a user is applying pressure to the region of interest; and
at least one image of the region of interest after the user releases the pressure from the region of interest.

15. The system of claim 14, wherein as part of processing the one or more images, the processing circuitry is configured to determine at least one of:
a change in size of tissue in the region of interest between the at least one image of the region of interest while the user is applying pressure to the region of interest and the at least one image of the region of interest after the user releases the pressure from the region of interest;
a change in color of the tissue in the region of interest between the at least one image of the region of interest while the user is applying pressure to the region of interest and the at least one image of the region of interest after the user releases the pressure from the region of interest;
a rate of change in the color of the tissue in the region of interest between the at least one image of the region of interest while the user is applying pressure to the region of interest and the at least one image of the region of interest after the user releases the pressure from the region of interest; or
a rate of change in a slope of a skin surface of the tissue in the region of interest between the at least one image of the region of interest while the user is applying pressure to the region of interest and the at least one image of the region of interest after the user releases the pressure from the region of interest.

16. The system of claim 1, wherein the system further comprises a flash, and wherein the one or more images comprise a video of light from the flash reflected back to the camera sensor when at least one of the flash or the camera sensor is in contact with the region of interest.

17. The system of claim 1, wherein the result comprises a prediction of worsening heart failure.

18. The system of claim 1, wherein the one or more images comprise a first one or more images, wherein the result is a first result, and wherein the processing circuitry is further configured to:
store the first one or more images in memory;
capture a second one or more images of the region of interest of the patient via the camera sensor after a time period;
compare the second one or more images to the first one or more images;
determine a second result based on the comparison, the second result being related to the heart failure status of the patient; and
generate a second indication for output regarding the second result.

19. A method of monitoring a heart failure status of a patient, the method comprising:
capturing, by processing circuitry, one or more images of a region of interest of the patient via a camera sensor;
processing, by the processing circuitry, the one or more images;
determining, by the processing circuitry, a result based on the processing, the result being related to the heart failure status of the patient; and
generating, by the processing circuitry, a first indication for output regarding the result.

20. Non-transitory computer-readable storage media storing instructions, which when executed, cause processing circuitry to:
capture one or more images of a region of interest of interest patient via a camera sensor;
process the one or more images;
determine a result based on the processing, the result being related to a heart failure status of the patient; and
generate a first indication for output regarding the result.
